(19) United States
(12) Patent Application Publication
(10) Pub. No.: US 2025/0186762 A1
(43) Pub. Date: Jun. 12, 2025

(54) BIOELECTRONIC SMART BANDAGE FOR CONTROLLING WOUND PH THROUGH PROTON DELIVERY

(71) Applicant: The Regents of the University of California, Oakland, CA (US)

(72) Inventors: Narges Asefifeyzabadi, Santa Cruz, CA (US); Tiffany Nguyen, Santa Cruz, CA (US); John Selberg, Santa Cruz, CA (US); Maryam Tebyani, Santa Cruz, CA (US); Prabhat Baniya, Santa Cruz, CA (US); Pattawong Pansodtee, Santa Cruz, CA (US); Mircea Teodorescu, Santa Cruz, CA (US); Marco Rolandi, Santa Cruz, CA (US)

(73) Assignee: The Regents of the University of California, Oakland, CA (US)

(21) Appl. No.: 18/849,414

(22) PCT Filed: Mar. 24, 2023
(86) PCT No.: PCT/US2023/016245

§ 371 Date: Sep. 20, 2024

Related U.S. Application Data
(60) Provisional application No. 63/323,321, filed on Mar. 24, 2022.

(51) Int. Cl. A61N 1/04; A61N 1/36

(52) U.S. Cl. CPC A61N 1/0428; A61N 1/0468; A61N 1/36014

(57) ABSTRACT

A bioelectronic smart device capable of charge (e.g., proton) delivery for providing treatment (e.g., changing the pH) of wound surfaces on in vitro and in vivo models. In one example, this is achieved through the use of a device that incorporates a hydrogel-based ion pump with a custom PCB for pump actuation. In one example, we were able to achieve delivery of approximately 6-19 nanomoles of protons to in vivo wound surfaces after ten minutes.

### CROSS REFERENCE TO RELATED APPLICATIONS

This application claims the benefit under 35 U.S.C. Section 119(e) of the following co-pending and commonly-assigned U.S. Provisional Application Ser. No. 63/323,321, filed on Mar. 24, 2022, by Narges Asefifeyzabadi and Marco Rolandi, entitled “BIOELECTRONIC SMART BANDAGE FOR CONTROLLING WOUND PH THROUGH PROTON DELIVERY,” Attorney's Docket Number 284.0011USP1; which application is incorporated by reference herein.

### STATEMENT REGARDING FEDERALLY SPONSORED RESEARCH AND DEVELOPMENT

This invention was made with Government support under Grant No. D20AC00003, awarded by the Defense Advanced Research Projects Agency (DARPA). The Government has certain rights in the invention.

### BACKGROUND OF THE INVENTION

### 1. Field of the Invention

The present invention relates to a smart bandage and methods of making the same.

### 2. Related Art

Ion pumping technologies demonstrated in the literature¹⁹⁻²⁴ require implanting the devices in animals to allow prolonged delivery of ions. Wearable wireless devices have also been demonstrated but only for intermittent delivery/signaling with less stringent power requirements²⁵⁻²⁷ or make use of traditional power supply^{28,29}. In contrast, wireless implants have allowed optogenetics experiments in moving mice^{30,31} but require the use of specialized cages for wireless power transfer. What is needed are wearable ion pump device for sustained delivery of treatment dose in freely moving patients without requiring specialized cages. The present invention satisfies this need.

### BRIEF DESCRIPTION OF THE DRAWINGS

Referring now to the drawings in which like reference numbers represent corresponding parts throughout:

FIG. 1A-1B. Wired in vivo device placed on the back of a anaesthetized mouse for short duration H+ delivery using an external voltage controller (FIG. 1A) and circuit diagram of wired PCB showing electrical connection to the PDMS and wound bed (FIG. 1B).

FIG. 2. The wired in vivo device. A) The underlying PDMS device with embedded wires and inserted contact pins. B) The complete CAD assembly of PCB mounted over PDMS. C) The exploded view of the wired in vivo device showing individual components/layers. D) The fabricated prototype of the wired in vivo device connected to a breakout board via a ribbon cable.

FIG. 3. The external voltage controller connected to the wired in vivo device. The controller is a PCB attached on top of a Raspberry Pi 3B+. All the major electronic components are highlighted in boxes.

FIG. 4. Device Fabrication Process, A) CAD Model-Main Body of PDMS Device and Side View. A 3D printer was used for fabrication of PDMS molds following filling with PDMS, baked for 48 h in oven. As shown in the figure, the device contains four reservoirs to hold interest substances which interact with the wound. B) top PDMS and mold (left), bottom PDMS and mold (middle), and bonded PDMS (right), wherein FIG. 4B(iv) is a zoomed in view of the right hand figure in FIG. 4B(iii). C) Top mold filled with PDMS (left), demolded top PDMS piece (middle), and wire insertion (right). D) Aluminum custom clamps are used to mechanically secure the two PDMS pieces with wires embedded in between. Shown are the PDMS top and bottom pieces in their respective clamps in the leftmost two images. The last three images show the assembled clamp, filling of bonded PDMS, and filled PDMS.

FIG. 5. A) Schematic of in vivo ion pump. B) A 0.5 mm diameter Ag wire is used as the working electrode and a 0.5 mm AgCl wire is used as the counter/reference electrode to load a 5 mm hydrogel-capillary. C) Typical current responses from 30-minute loading with H⁺. Steady-state current is around 5 microamps.

FIG. 6. A) Current profile of device for in vitro test (2V). Current profiles from actuation of the three devices on anesthetized mouse wounds for Days 0 and 1. B) Current responses on Day 0, where 1.5 V was applied across each device for 8 minutes. C) Current responses on Day 1, where 2 V was applied across each device for 10 minutes.

FIG. 7. A) Wired in vivo device placed on the back of a chicken breast for short duration H+ delivery using an external voltage controller. B) Current response on Days 0, 1, 2, and 3, where 2 V was applied across working electrode (WE) and reference electrode (RE) for 60 s, followed by 0 V application.

FIG. 8. A) Current responses on Day 0, where 1.5 V was applied across WE and RE on each device for 8 minutes. B) Total charge and accumulated dose on Day 0. C) Current responses on Day 1, where 2 V was applied across WE and RE on each device for 10 minutes. D) Total charge and accumulated dose on Day

FIG. 9. A) An example of IHC staining on a tissue sample where five regions were sampled. B) Representative M1 and M2 macrophage staining at wound centers from control and H+-treated mice. C) M1/M2 ratio of the H+-treated wounds is 35.86% lower compared to the control wound.

FIG. 10. Flowchart illustrating a method of making a device.

FIG. 11. Flowchart illustrating a method of operating a device.

FIG. 12. Hardware environment for controlling the device.

### SUMMARY OF THE INVENTION

A bioelectronic smart device capable of charge (e.g., proton) delivery for providing treatment (e.g., changing the pH) of wound surfaces on in vitro and in vivo models. In one example, this is achieved through the use of a device that incorporates a hydrogel-based ion pump with a custom PCB for pump actuation. In one example, we were able to achieve delivery of approximately 6-19 nanomoles of protons to in vivo wound surfaces after ten minutes.

Example embodiments include, but are not limited to, the following.

1. A device, comprising:
a wearable ion pumping system pumping ions to and from a treatment site so as to deliver and measure a dose of the ions treating the treatment site.

2. The device of example 1, wherein the wearable ion pumping system further comprises a control circuit activating the pumping and measuring the dose.

3. The device of example 2, wherein the wearable ion pumping system further comprises:
a housing comprising one or more reservoirs storing a fluid comprising the ions;
a system of conduits connected to the housing, the conduits loaded with a fluid between the reservoir and the treatment site; and
a plurality of electrodes electrically connected to the fluid in the reservoirs and the control circuit, wherein the control circuit activates the pumping by applying one or more voltages to the fluid in the reservoirs via the electrodes.

4. The device of example 3, wherein the control circuit comprises a potentiostat applying one or more voltages and sensing one or more currents flowing through the treatment site.

5. The device of example 3, wherein:
the electrodes each comprise a pin comprising a first end electrically coupled to a wire,
the housing consists essentially of a polymer and comprises:
   molded cavities comprising the reservoirs;
   through holes through which the pins are inserted, so that the first end of each of the pins electrically connects to the wire in a different one of the reservoirs; and
   mounts comprising openings through which each of the conduits are inserted and mounting a first conduit end of each of the conduits in fluidic connection with a different one of the reservoirs, so that a second end of the each of the conduits can be in physical contact with the treatment site; and
the device further comprises a printed circuit board soldered to a second end of the pins, wherein the printed circuit board is electrically connected to the control circuit.

6. The device of example 5, wherein:
the housing comprises:
   a top (printed circuit board side) part comprising the molded cavities and the through holes and physically attached to the printed circuit board side via the pins; and
   a bottom (treatment site side) part comprising the mounts and a lid enclosing the molded cavities to form the reservoirs; and the conduits comprise capillaries.

7. The device of example 5, wherein the polymer consists essentially of PDMS.

8. The device of example 5, wherein:
the reservoirs comprise one or more first reservoirs and one or more second reservoirs;
the ions comprise first polarity ions and second polarity ions; and
the control circuit applies:
   the voltage comprising a first polarity bias to the fluid via the electrodes in the first reservoirs to drive the first polarity ions to the treatment site through a first one of the conduits; and
   the voltage comprising a second polarity bias to the fluid via the electrodes in the second reservoirs to pull the second polarity ions from the treatment site through a second one of the conduits, to maintain charge balance.

9. The device of example 8, wherein the control circuit measures a current comprising the ions to determine the dose of ions delivered to the treatment site.

10. The system of example 5, wherein the wires are embedded in the polymer and in fluidic connection with the fluid in the reservoir.

11. The device of example 10, wherein the wires are connected to the pins via a conductive paste and the wires extend across a majority of a length or width of the reservoir.

12. The device of example 3, wherein the control circuit comprises multiple channels, each of the channels connected to a different one of the voltages and comprising one of the conduits delivering the ions in response to the one of the voltages and a current sense resistor in series with the voltage for sensing the current in the each of the channels.

13. The device of example 3, further comprising an electrical connector, wherein the control circuit is remotely connected to the electrodes with wiring via a break out board and the electrical connector.

14. The device of example 3, wherein the ions comprise H+ ions.

15. The device of example 14, wherein the fluid in the conduits comprises a hydrogel and the fluid in the reservoirs comprises a solution comprising the ions.

16. The device of example 2, wherein the ion pumping system comprises a microfluidic system pumping a fluid comprising the ions.

17. The device of example 2, wherein the ions control a pH of the treatment site.

18. The device of example 17, wherein the control circuit controls the ions to lower the pH so as to transition macrophages in the treatment site to an anti-inflammatory pro-reparative phonotype (away from an inflammatory phenotype) early in the treatment cycle.

19. A system comprising the device of example 2 coupled to a camera forming images of the wound, wherein the control circuit provides closed loop control of the pumping based on healing of the wound observed in the images.

20. The device of example 1, wherein the charge pumping system pumps biomolecules comprising the ions.

21. The device of example 1, wherein the device comprises a notch or protrusion for attaching the device to the treatment site.

22. The device of example 21, wherein the treatment site comprises a wound and the ions accelerate healing of the wound.

23. The device of example 1, further comprising a bandage, dressing, or patch attaching the device to the treatment site and covering the treatment site.

24. The device of example 1, further comprising an adhesive or mechanism for attaching the device to the treatment site.

### DETAILED DESCRIPTION OF THE INVENTION

In the following description of the preferred embodiment, reference is made to the accompanying drawings which form a part hereof, and in which is shown by way of illustration a specific embodiment in which the invention may be practiced. It is to be understood that other embodiments may be utilized and structural changes may be made without departing from the scope of the present invention.

### Technical Description

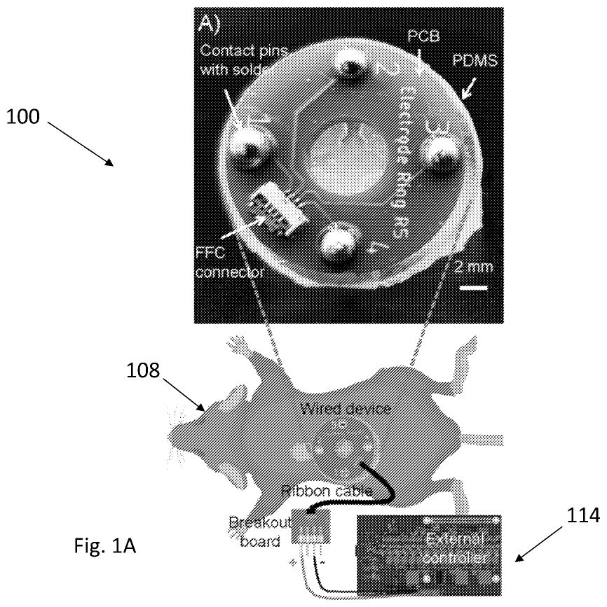

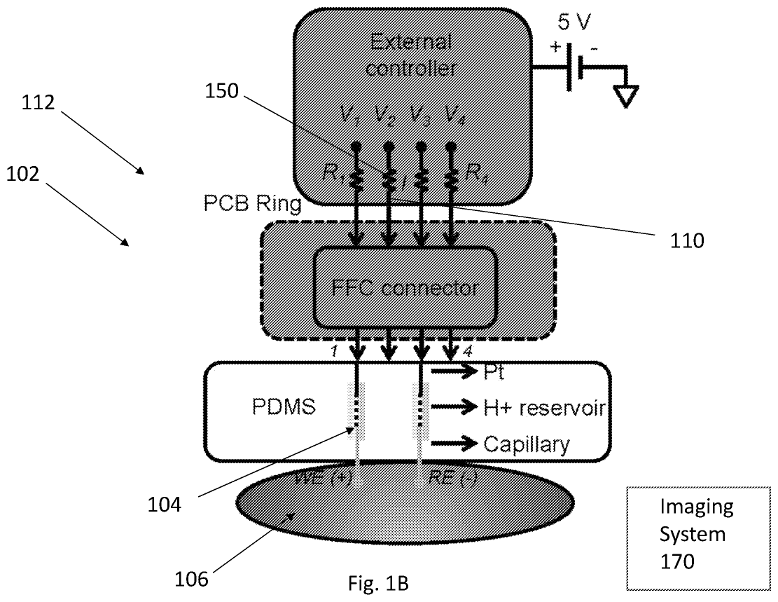

FIG. 1A and FIG. 1B illustrate a device/bandage 100, comprising a wearable charge pumping system 102 pumping charged species 104, comprising biomolecules or ions, to and from a treatment site 106 on living tissue 108 (e.g., of an animal or human), so as to deliver a dose of the charged species 104 treating the treatment site and collect an electrical signal 110 used to measure the dose. The wearable pumping system further comprises a control circuit 112 activating the pumping and measuring the dose. In the example shown, the control circuit 112 comprises a potentiostat 114 applying the voltages and sensing a current flowing through the treatment site.

### Example Wired Device

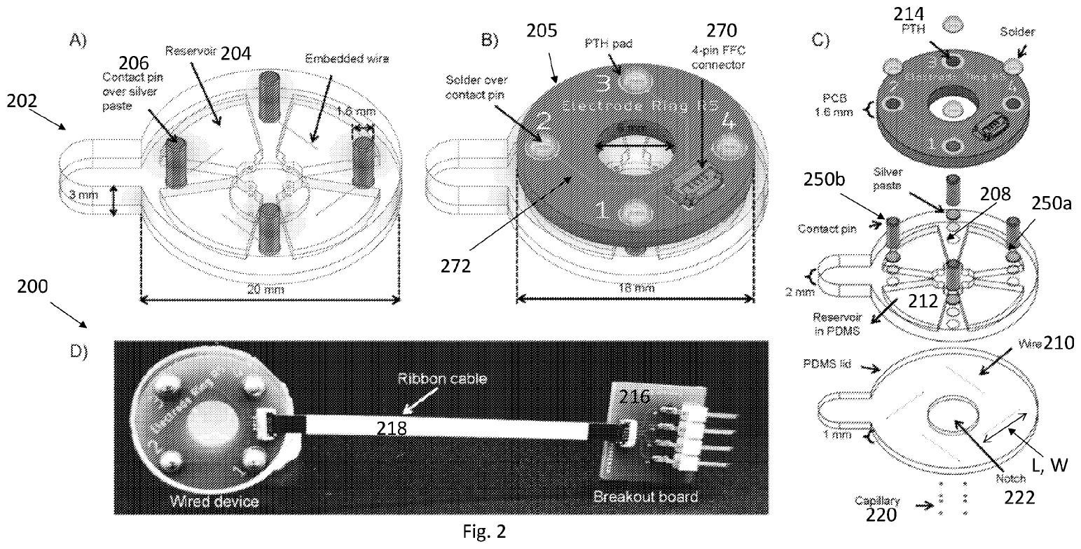

FIG. 2 illustrates an example wired device 200. The mechanical and electrical integration between the housing 202 (containing the ion reservoirs 204) and the control circuit PCB 205 is achieved through four contact pins 206 of 1.6 mm diameter and 4.8 mm length. The pins are located at a radial distance of 7 mm from the center. The pins are made of low-alloy steel and are chosen because of their availability and good solderability (comparable to that of copper). FIG. 2A illustrates the pins sit between the reservoirs 204 within the PDMS housing 202. The reservoirs hold 0.5 M HCl solution 212 for delivery of H+ to the wound. The bottom half of the pins are manually coated with conductive (silver) epoxy paste before insertion into the four holes 208 in the PDMS device. This ensures that the pins make strong electrical connection with the embedded Pt wires 210 underneath as well as strong adherence/bonding to the PDMS hole walls. FIGS. 2B and 2C illustrate the PCB 205 comprises a ring including four plated through holes (PTHs) 214 of matching diameter. The PCB ring is placed flush on PDMS by simply guiding the ring's PTHs over the pins. The PCB ring houses a side-entry, 0.5 mm pitch, flexible flat cable (FFC) connector.

The fabricated device (PCB ring integrated with PDMS) weighs 2 g and is connected to a breakout board 216 via a ribbon cable 218, as shown in FIG. 2D. The protruding top portion of the pins (see FIG. 2A for reference) are soldered to the pads of the PTHs using soldering iron to ensure a strong electrical/mechanical connection and fully secure the ring to the PDMS, as shown in FIG. 2B. Four H+ loaded capillary tubes 220 (inner diameter 100 μm, outer diameter 375 μm) provide paths for the H+ from the reservoirs to the wound bed. The capillary tubes 220 of 2 to 3 mm length are inserted (within the 6 mm central region) into the bottom PDMS notch 222 and pushed into the reservoirs that are located in the top PDMS piece, as shown in FIG. 2C.

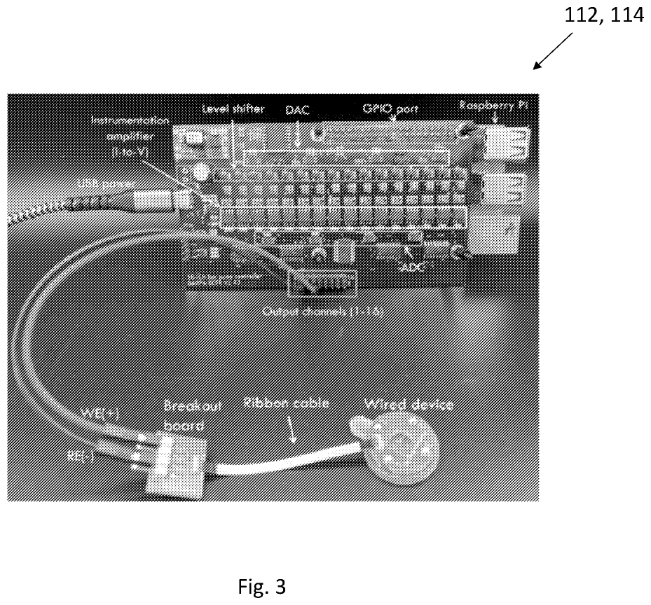

FIG. 3 illustrates the external voltage controller comprising control circuit 112 and potentiostat 114 connected to the wired device. The controller comprises an amplifier, current to voltage converter, level shifter, digital to analog converter (DAC), GPIO port, computer (raspberry pi), analog to digital converter and output channels.

### Example Housing and Fabrication Method

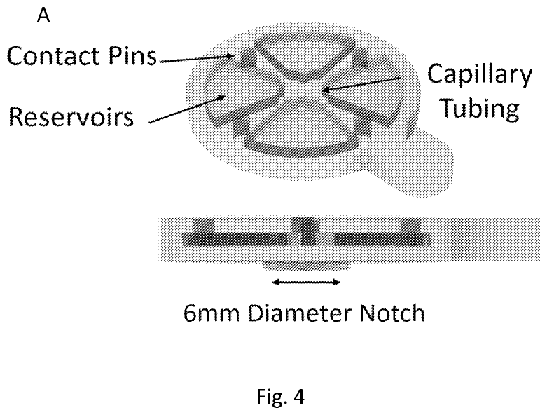

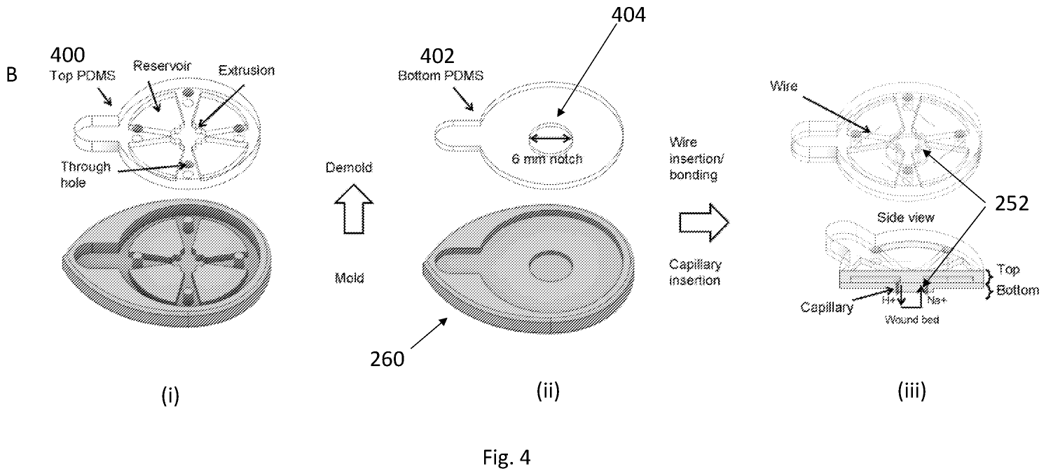

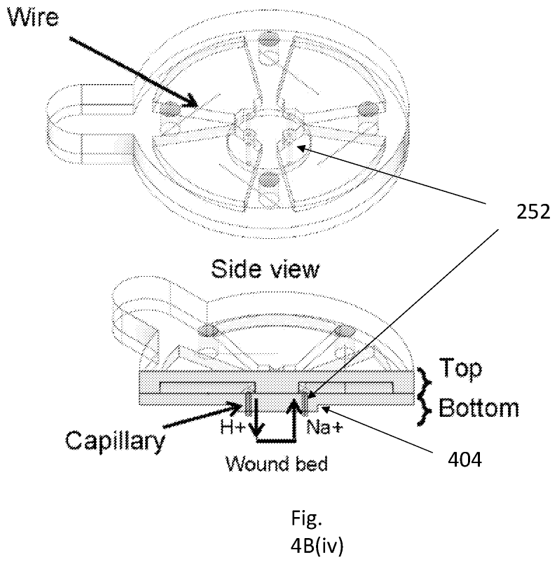

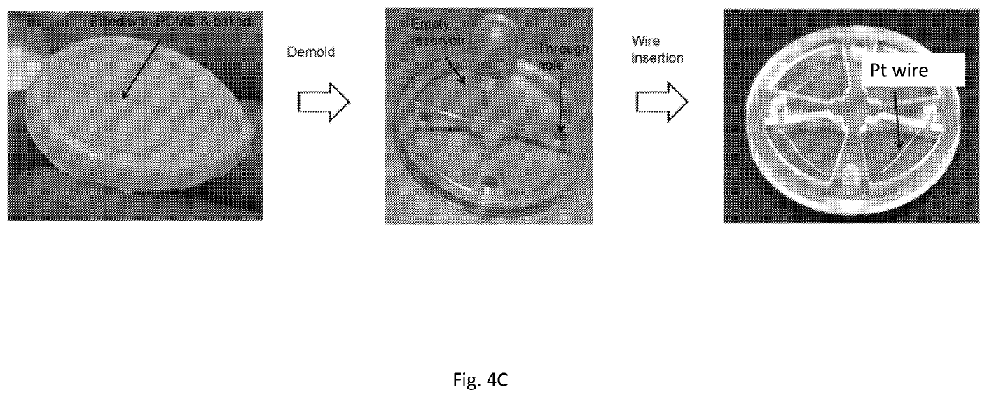

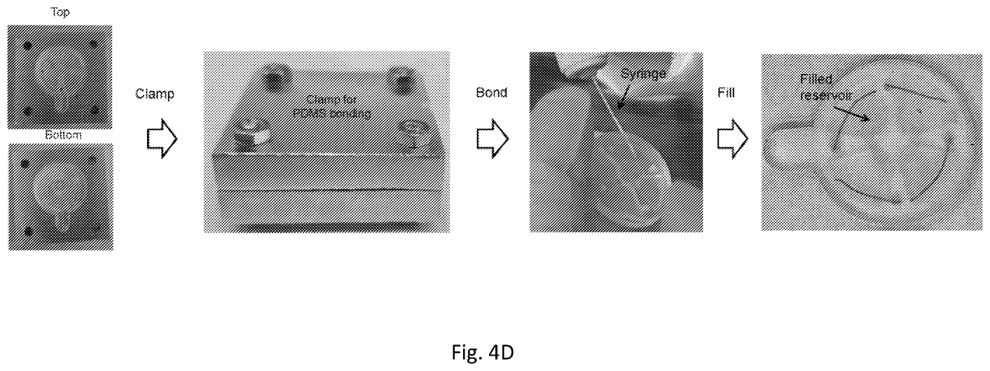

FIGS. 4A-4D illustrate an example housing PDMS fabrication process, wherein FIG. 4B illustrates method begins by 3D printing two-part molds that are sonicated in IPA, cleaned with water, dried with N₂, and then UV cured. Then the molds are filled with PDMS and baked for 48 h in an oven at 60° C. FIG. 4C illustrates the two PDMS pieces are then demolded by running a sharp blade along the edges (left and middle, as shown for the top piece). The top PDMS piece 400, shown as a CAD model in FIG. 4B (left), contains the reservoirs, through-holes for electronics interfacing, and an extruded feature to hold hydrogel-filled capillary tubes which interface with the wound surface. The bottom PDMS piece 402, as shown in FIG. 4B (middle), functions as a lid which seals the exposed face of the reservoirs and features a 0.5 mm tall, 6 mm diameter notch designed to sit in the wound. FIG. 4C (right) illustrates the method further comprises embedding Pt wires between the two pieces before bonding for electronics interfacing. The PDMS pieces are bonded by first activating the PDMS surfaces in an O₂ chamber, and then using custom aluminum clamps for mechanical contact and alignment, as shown in FIG. 4D (left three images). Devices are inspected to ensure successful bonding and wire placement, then the reservoirs are filled using a syringe prepared with 0.5 M HCl, as shown in FIG. 4D (right two images). Finally, the devices are parylene coated (with thickness of 2.561 μm) to mitigate substance leakage and bubble formation in the reservoirs.

### Example Application: Acceleration of Wound Healing

Acceleration of the wound-healing process has consistently been a major health care concern, especially for chronic wounds such as diabetic foot ulcers. To this end, altering wound pH has received much attention for clinical applications. It has been proven that decreasing the pH of alkaline wound surfaces to a pH range of 6.0-6.5 can expedite healing rates and the wound closure process [39].

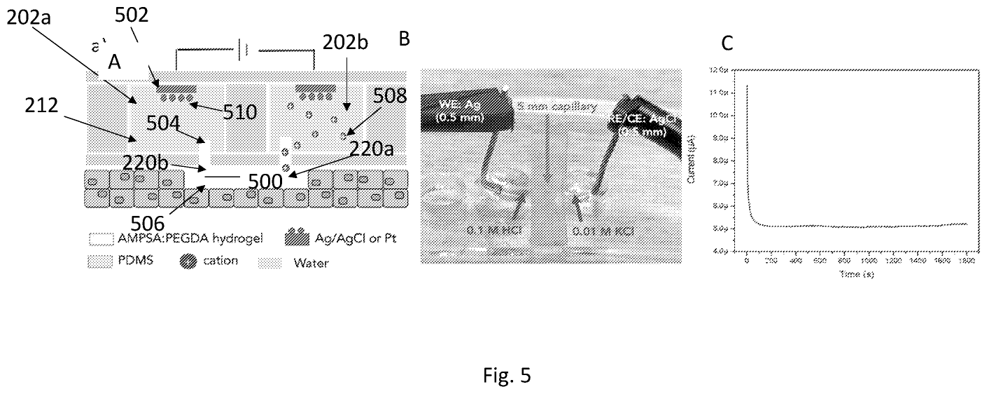

FIG. 5 illustrates a bioelectronic smart bandage capable of proton delivery for changing the pH of wound surfaces on in vitro and in vivo models. Protons are delivered on the wound surface by applying voltage across the working and reference electrodes which drives protons through the cation selective ion pump. The results in FIG. 6 indicate the bandage device can be used for longer periods of proton delivery to further manipulate wound pH and expedite wound healing. Thus, through the use of a device that incorporates a hydrogel-based ion pump with a custom PCB for pump actuation, delivery of approximately 6-19 nanomoles of protons through the hydrogel 500 (see FIG. 5) to in vivo wound surfaces after ten minutes was achieved.

### Example H+ Delivery Using the Example Wired Device

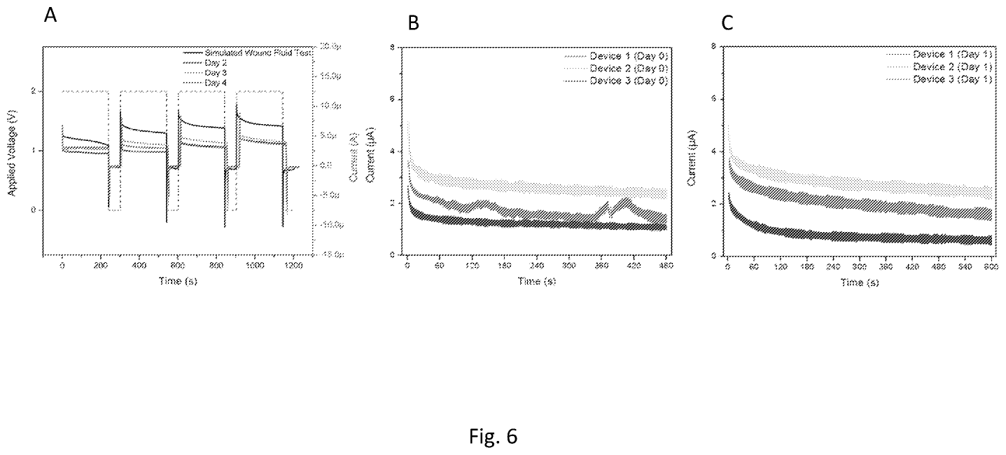

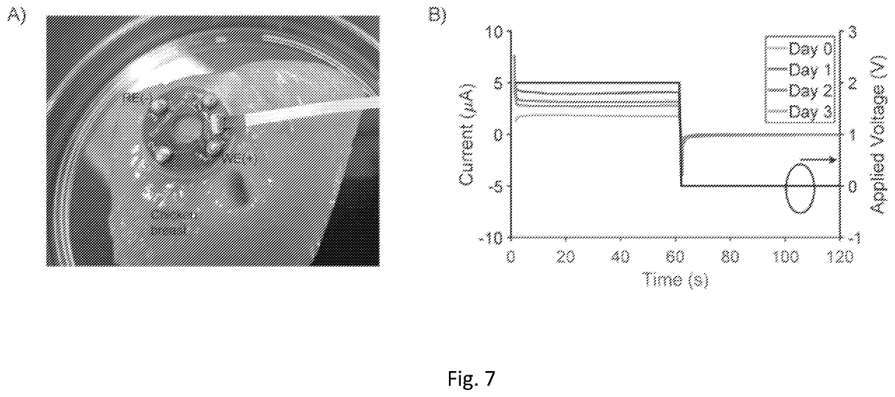

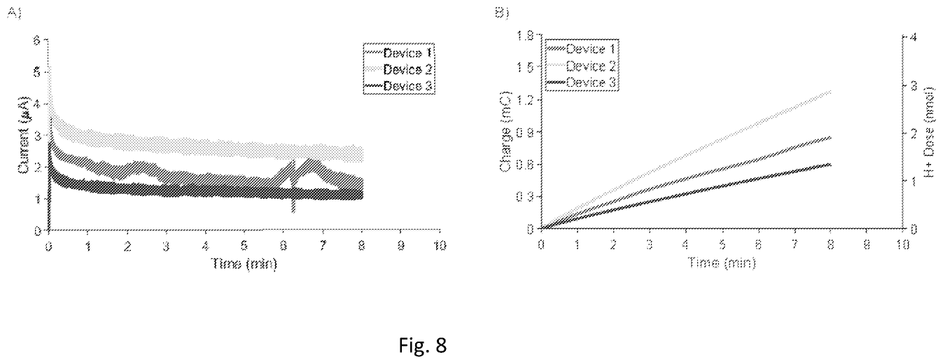

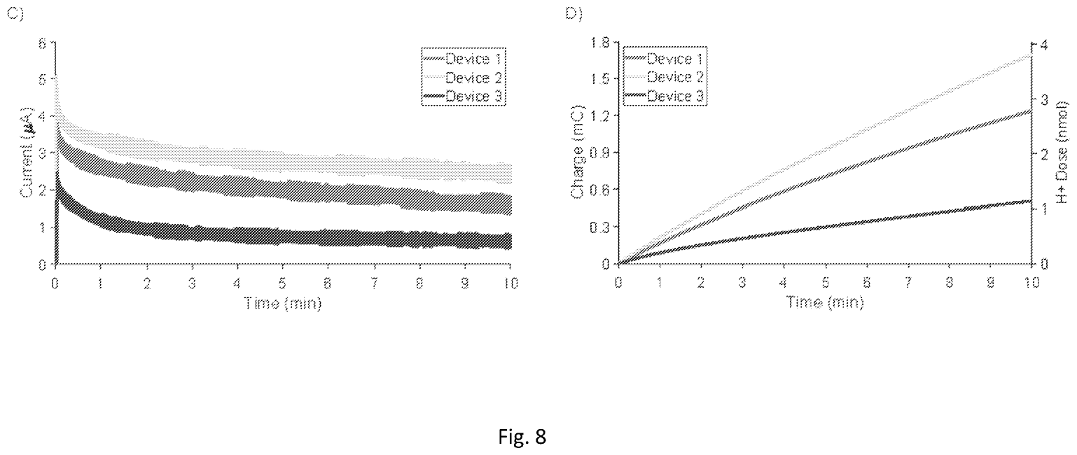

The bandage of FIG. 1 and FIG. 2 was used to deliver H+ (i.e., protons), e.g., for targeting macrophage recruitment. Actuation of the device using an applied voltage across Pt working electrode (WE) and reference electrode (RE) drives H+ through the cation-selective 2-acrylamido-2-methyl-1-propanesulfonic acid (AMPSA)-polyethylene glycol diacrylate (PEGDA) polyanion hydrogel in the device capillaries. Without being bound by a particular scientific theory, H+ exists in 0.5 M HCl solution mainly as H+ and Cl−; for a positive WE voltage, H+ is pushed from the reservoir containing the WE due to the Coulomb force, and to maintain charge balance, endogenous cations, primarily Na+⁴¹⁻⁴³, present in the tissue, are pulled into the reservoir containing the RE, as depicted in FIG. 8A (right). The counter-ion and endogenous anions, primarily Cl−, also contribute to the total current. H+ can thus be delivered to the target wound bed, and an approximate delivered concentration can be estimated from the resulting current. Devices were initially tested on chicken breast (as it closely emulates the wound impedance) to determine if the current levels are high enough for H+ delivery using the external voltage controller (see FIG. 3 and FIG. 7). As depicted in FIG. 1, the current in each channel is measured by the controller using current sense resistors Rᵢ=1 kΩ±0.1%.

Over a four-day experiment, devices were affixed to mouse wounds, actuated for H+ delivery, and checked for robustness. Three mice were used; two circular 6 mm splinted excisional wounds were created on each. One wound was used for control testing, consisting of plain PDMS device bodies with capillaries (without PCBs, reservoirs unfilled). The other wound was used for actuated device testing, consisting of attached PCBs and reservoirs filled with 0.5 M HCl. FIG. 1 depicts the approximate location of the actuated device on the mouse under which wound is created. The control device is placed opposite (i.e., above) the actuated device.

On Day 0 of the experiment, wounds were surgically generated and images were captured. This was followed by an 8 min actuation of each actuated device using the external voltage controller wired to the PCB of the bandage, as illustrated in FIG. 1A. Actuation duration was limited by the allowable anesthesia time for the mice. Devices were secured to the mouse wounds using Tegaderm. On Day 1, mice were re-anesthetized, and devices were actuated for 10 min. Resultant current responses, total charge, and accumulated dose from the device actuation across the two days are shown in FIG. 8.

The total charge Q in Coulomb (C) at time t can be calculated by integrating current response I over a time period of
(t−tₛₜₐᵣₜ) as

$$Q ( t ) = {\int }_{{t}_{start}}^{t} I ( τ ) d τ ( C )$$

The dose D(t) in mol can be calculated as

$$D ( t ) = \frac{η Q ( t )}{F} ( mol )$$

where η is the average H+ delivery efficiency and F=96485.3321 C/mol is the Faraday's constant.

The endogenous Na+ and Cl− ions compete with H+ ions to carry the current⁴³. The H+ delivery efficiency was determined to be η=21.7% across all devices using the method utilized by Dechiraju et al.⁴⁴. Based on equation (1), the current responses in FIGS. 8A and C are integrated over time in MATLAB using the cumulative trapezoidal method. Then using equation (2), we found that around 1.91, 2.86, and 1.35 nanomoles of H+ were delivered using three separate devices on Day 0 whereas 2.79, 3.82, and 1.13 nanomoles of H+ were delivered on Day 1, as shown in FIG. 8B and D. Devices were not actuated after Day 1, as lowering pH past the inflammation stage of wound healing may impede the re-epithelialization of the wound. On Day 2 of the experiment, devices were visually inspected to ensure no interference from the mice had occurred. On Day 3, mice were sacrificed, wound images were again captured, and wound tissue was harvested for IHC staining. The accumulated dose delivered by the devices for each day is summarized in Table 1.

pH change calculations were performed in a local delivery area of 5 μL, after which H+ would diffuse to the remainder of the wound bed. The buffering capacity of wound fluid was assumed to be near that of blood at physiologically relevant pH levels, 38.5 mEq/L/pH⁴⁵. This experiment was performed to lower the local pH (assuming an initial wound pH of around 7)⁴⁶. pH estimations are shown in Table 1.

TABLE 1
H+ dose delivered to the wounds by wired
devices and local pH changes on Day 0 and Day 1.

| Day | Device | H+ Dose D (nmol) | Final pH |
|---|---|---|---|
| Day 0 | 1 | 1.91 | 6.99 |
| | 2 | 2.86 | 6.99 |
| | 3 | 1.35 | 6.99 |
| Day 1 | 1 | 2.79 | 6.99 |
| | 2 | 3.82 | 6.98 |
| | 3 | 1.13 | 6.99 |

### M1/M2 Macrophage IHC Staining Results

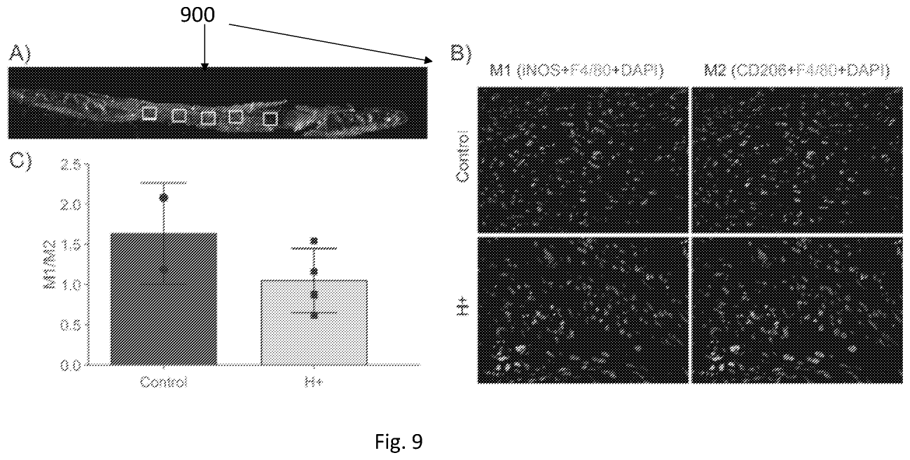

The IHC staining was carried out on the two control and four H+-treated mice. M1 and M2 macrophages were manually counted based on the double-positive staining with blind evaluation. For each mouse, five adjacent regions at the wound center were imaged at 40× magnification for macrophage quantification, as shown in FIG. 9A. A set of typical stained slice for the tissues (at wound centers) gathered for a control and a H+-treated mice, is shown in FIG. 8B. After analyzing the data, the staining results show that on average, M1/M2 ratio of the H+-treated wounds is 35.86% lower compared to the control wound, as shown in FIG. 9C. All data are presented as mean±standard deviation. On average, the inflammatory phase is shortened in the treatment group as measured by the decrease in the M1/M2 ratio.

### Device and System Embodiments

Illustrative embodiments of the inventive subject matter described herein (e.g., device, apparatus, system, or method) include, but are not limited to, the following examples (referring also to FIGS. 1-12).

1. A system, apparatus, or device 100, comprising:
a wearable ion pumping system 102 pumping charged species 104 (e.g., ions, including positive and/or negative ions, or biomolecules) to and from a treatment site 106 so as to deliver and measure a dose of the charged species treating the treatment site (e.g., living tissue).

2. The device of example 1, wherein the wearable ion pumping system further comprises a control circuit 112 activating the pumping and measuring the dose.

3. The device of example 1 or 2, wherein the wearable ion pumping system further comprises:
a housing 202 comprising one or more reservoirs 204 storing a fluid (e.g., first fluid) 212 comprising the charged species 104 (e.g., ions);
a system of conduits 220 (e.g., capillaries) connected to the housing, the conduits loaded with a fluid (e.g., second fluid 500) between the reservoir and the treatment site;
a plurality of electrodes 502 electrically connected to the first fluid 212 and the control circuit 112, wherein in the control circuit 112 activates the pumping by applying one or more voltages to the first fluid 212 via the electrodes 502.

4. The device of example 2 or 3, wherein the control circuit 112 comprises a potentiostat applying the one or more voltages and sensing one or more currents flowing through the treatment site (e.g., in response to the one or more voltages).

5. The device of example 3, wherein:
the electrodes 502 each comprise a pin 206 comprising a first end electrically coupled to a wire 210,
the housing 202 consists essentially of a polymer and comprises:
   molded cavities comprising the reservoirs 204;
   through holes 208 through which the pins 206 are inserted, so that the first end 250a of each of the pins electrically connects to the wire 210 in a different one of the reservoirs, and
   mounts 252 comprising openings through which each of the conduits 220 are inserted and mounting a first conduit end 504 of each of the conduits in fluidic connection with a different one of the reservoirs 204, so that a second end 506 of the each of the conduits 220 can be in physical contact with the treatment site (e.g., securely engaged with, securely sitting in, or securely attached to, or embedded in the treatment site or wound bed); and
the device further comprises a printed circuit board 205 soldered to a second end 250b of the pins, wherein the printed circuit board comprises a connection/connector 270 for electrical connection to the control circuit 112. In various examples, the printed circuit board comprises wiring/conductive tracks 272 connecting the connector to the pins 206 and/or acts as an electrical interface between the housing/fluid/treatment site and the control circuit.

6. The device of example 5, wherein the housing 202 comprises:
a top (printed circuit board side) part 400 comprising the molded cavities and the through holes 208 and physically attached to the printed circuit board 205 via the pins 206,
a bottom (treatment site side) part 402 comprising the mounts 252 and a lid 260 enclosing the molded cavities to form the reservoirs 202,
the conduits 220 comprise capillaries.

7. The device of example 5, wherein the polymer consists essentially of, or comprises, Polydimethylsiloxane (PDMS) or a silicone.

8. The device of example 5, wherein:
the reservoirs 202 comprise one or more first reservoirs 202a and one or more second reservoirs 202b,
the ions comprise first polarity ions 508 (e.g., H+) and second polarity ions 510 (e.g., Cl−),
the control circuit 112 applies:
the voltage comprising a first polarity bias to the fluid 212 via the electrodes 502 in the first reservoirs to drive the first polarity ions 508 to the treatment site through a first one of the conduits 220a,
the voltage comprising a second polarity bias to the fluid via the electrodes 502 in the second reservoirs to pull the second polarity ions 510 from the treatment site through a second one 220b of the conduits, to maintain charge balance.

9. The device of any of the examples 1-8, wherein the control circuit 112 measures a current comprising the ions to determine the dose of ions (first polarity ions) delivered to the treatment site.

10. The device of any of the examples 2-9, wherein the control circuit comprises multiple channels, each of the channels comprising one of the conduits 220 delivering the charged species (e.g., ions) in response to an applied bias (V₁, V . . . V₄ . . . Vn) and a current sense resistor 150 in series with the applied bias for sensing the current in the channel.

11. The device of any of the examples 2-10, wherein the control circuit 112 is remotely connected to the electrodes 502 with wiring via a break out board 216 and a connector 205/218. In one or more examples, the device comprises a printed circuit board 205 (e.g., attached to housing 202) comprising a connector 270 for connecting to the control circuit. In one or more examples, printed circuit board 205 comprises conductive tracks 272 connecting the connector to the pins 206/electrodes 502.

12. The device of example 2, wherein the ions control a pH of the treatment site.

13. The device of example 12, wherein the control circuit 112 controls the ions to lower the pH so as to transition the macrophages to an anti-inflammatory pro-reparative phonotype (away from an inflammatory phenotype) early in the treatment cycle.

14. The device of example 3, wherein the ions comprise H+ ions.

15. The device of example 14, wherein the fluid (e.g., second fluid 500) in the conduits 220 comprises a hydrogel and the fluid (e.g., first fluid 212) in the reservoirs 202 comprises a solution comprising the ions.

16. The device of example 5, wherein the wires 210 are embedded in the polymer and in fluidic connection and/or electrical contact with the fluid (e.g., first fluid 212) in the reservoir 214.

17. The device of example 15, wherein the wires 210 are connected to the pins 206 via conductive paste and the wires extend across a majority of a length L or a width W of the reservoir 214.

18. The device of any of the examples 1-17 coupled to an imaging system 170 imaging the wound, for closed loop control of the delivery based on healing of the wound observed in one or more images 900 of the wound obtained using the imaging system.

19. The device of any of the examples 1-18, wherein the ion pumping system 100 comprises a microfluidic system pumping a fluid comprising the ions.

20. The device of any of the examples 1-19, wherein the ion pumping system pumps biomolecules comprising the ions.

21. A system comprising the device of any of the examples 1-20 coupled to a camera forming images of the wound, wherein the control circuit 112 (coupled to the camera) provides closed loop control of the pumping based on healing of the wound observed in the images.

22. The device of any of the examples 1-21, wherein the device comprises a notch or protrusion 404 for attaching or engaging the device to the treatment site, or sitting or mounting the device 100 or embedding conduits 202 in the treatment site.

23. The device of any of the examples 1-22, wherein the treatment site comprises a wound and the ions accelerate healing of the wound.

24. The device of any of the examples 1-23, further comprising a bandage, dressing, compress, balm, or patch attaching the device to the treatment site and covering and/or protecting the treatment site. Example bandages include, but are not limited to, a strip of material used to bind a wound or to protect an injured part of the body, a strip of fabric used especially to cover, dress, and bind up wounds, or a flexible strip or band used to cover, strengthen, or compress something.

25. The device of any of the examples 1-24, further comprising an adhesive or mechanism or means for attaching the device to the treatment site, and equivalents thereof.

26. A bandage for delivering positive/negative ions via ion pump control circuit 112.

27. The device of any of the examples 1-27, wherein the control circuit 112 comprises a potentiostat (as illustrated in FIG. 3) comprising:
one or more channels for electrically connected to the electrodes (WE, RE, 502);
an output stage comprising a digital to analog converter (DAC) circuit and a first level shifter circuit, wherein the first level shifter circuit is electrically connected to one or more of the channels and the DAC circuit; and
an input stage comprising a current to voltage converter circuit electrically connected to one or more of the channels; and an analog to digital converter (ADC) circuit
wherein:
the first level shifter circuit at least scales or shifts a DAC voltage output from the DAC circuit to the voltage comprising a desired level applied between the electrodes; and
the current to voltage converter optionally comprising a shunt resistor connected to an amplifier;
a computer comprising one or more processors; one or more memories; and one or more applications stored in the one or more memories, wherein at least one of the applications executed by the one or more processors:
controls the potentiostat to configure and set the voltages applied to the electrodes and/or receive current measurements outputted from the electrodes in response to the voltages.

28. The device of example 27, further comprising a second level shifter circuit connected to a first output of the current to voltage converter circuit; and the analog to digital converter (ADC) circuit connected to a second output of the second level shifter circuit.

29. The device of any of the examples, wherein the control circuit 112 comprises one or more channels comprising a voltage source in series with a resistor.

30. The device 100 of any of the examples 1-29, wherein the control circuit 112 (optionally comprising potentiostat) 114 is connected to the housing via flexible wire.

31. The device 100 of example 30 wherein the control circuit 112, 114 is on a printed circuit board attachable (using adhesive, tape, or other means for attaching) to a body of a human or animal having the treatment site 106.

32. The device of any of the examples, wherein the control circuit comprises a potentiostat comprising, or based on, the potentiostat in [32].

### Example Process Steps

a. Fabrication

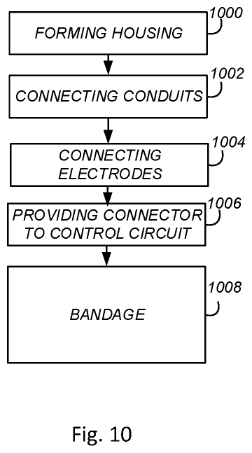

FIG. 10 illustrates a method of making a device according to one or more embodiments.

Block 1000 represents forming a housing comprising reservoirs for storing a fluid (e.g., first fluid) comprising ions.

Block 1002 represents connecting a system of conduits connected to the housing, the conduits may be loaded with a fluid (e.g., second fluid).

Block 1004 represents electrically connecting a plurality of electrodes 502 to the fluid (e.g. first fluid) in the reservoirs.

Block 1006 represent providing a connector for connecting the electrodes to a control circuit for activating the pumping of the ions by applying a voltage to the fluid via the electrodes.

Block 1008 represents the end result, e.g., as illustrated in FIG. 1A. The device can be fabricated and implemented in a number of ways, including but not limited to, the examples 1-32 described above in the section entitled “Device and System Embodiments”.

b. Method of Operation

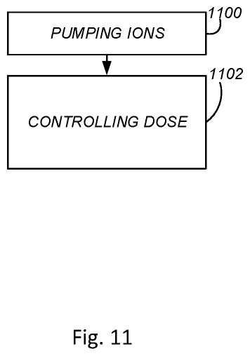

FIG. 11 illustrates a method of applying charged species (e.g., ions) to a treatment site.

Block 1100 represents attaching a device to a treatment site, wherein the device pumps ions from a reservoir to a treatment site.

Block 1102 represents controlling a dose of the ions to accelerate healing of the treatment site, using a control circuit.

The method can be implemented using the device of any of the examples described herein, including but not limited to the examples 1-32 in the section entitled “Device and System Embodiments”.

### Example Hardware Environment

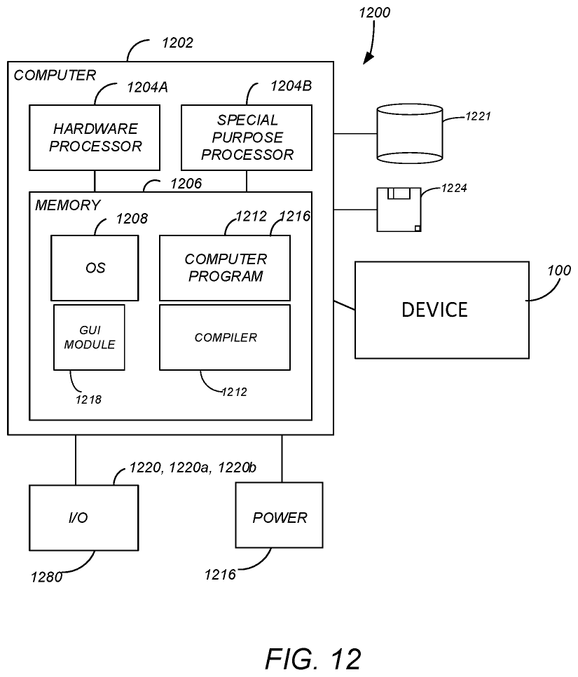

FIG. 12 is an exemplary hardware and software environment 1200 (referred to as a computer-implemented system and/or computer-implemented method) used to implement one or more embodiments of the invention (e.g., control and/or read out results of the multi-channel potentiostat 200). The hardware and software environment includes a computer 1202 and may include peripherals. Computer 1202 may be a user/client computer, server computer, or may be a database computer. The computer 1202 comprises a hardware processor 1204A and/or a special purpose hardware processor 1204B (hereinafter alternatively collectively referred to as processor 1204) and a memory 1206, such as random access memory (RAM). The computer 1202 may be coupled to, and/or integrated with, other devices, including input/output (I/O) devices such as a keyboard 1214, a cursor control device 1216 (e.g., a mouse, a pointing device, pen and tablet, touch screen, multi-touch device, etc.) and a printer 1228. In one or more embodiments, computer 1202 may be coupled to, or may comprise, a portable or media viewing/listening device 1232 (e.g., an MP3 player, IPOD, NOOK, portable digital video player, cellular device, personal digital assistant, etc.). In yet another embodiment, the computer 1202 may comprise a multi-touch device, mobile phone, gaming system, internet enabled television, television set top box, or other internet enabled device executing on various platforms and operating systems.

In one embodiment, the computer 1202 operates by the hardware processor 1204A performing instructions defined by the computer program 1210 (e.g., controlling potentiostat) under control of an operating system 1208. The computer program 1210 and/or the operating system 1208 may be stored in the memory 1206 and may interface with the user and/or other devices to accept input and commands and, based on such input and commands and the instructions defined by the computer program 1210 and operating system 1208, to provide output and results.

Output/results may be presented on the display 1222 or provided to another device for presentation or further processing or action. In one embodiment, the display 1222 comprises a liquid crystal display (LCD) having a plurality of separately addressable liquid crystals. Alternatively, the display 1222 may comprise a light emitting diode (LED) display having clusters of red, green and blue diodes driven together to form full-color pixels. Each liquid crystal or pixel of the display 1222 changes to an opaque or translucent state to form a part of the image on the display in response to the data or information generated by the processor 1204 from the application of the instructions of the computer program 1210 and/or operating system 1208 to the input and commands. The image may be provided through a graphical user interface (GUI) module 1218. Although the GUI module 1218 is depicted as a separate module, the instructions performing the GUI functions can be resident or distributed in the operating system 1208, the computer program 1210, or implemented with special purpose memory and processors.

In one or more embodiments, the display 1222 is integrated with/into the computer 1202 and comprises a multi-touch device having a touch sensing surface (e.g., track pod or touch screen) with the ability to recognize the presence of two or more points of contact with the surface. Examples of multi-touch devices include mobile devices (e.g., IPHONE, NEXUS S, DROID devices, etc.), tablet computers (e.g., IPAD, HP TOUCHPAD, SURFACE Devices, etc.), portable/handheld game/music/video player/console devices (e.g., IPOD TOUCH, MP3 players, etc.), touch tables, and walls (e.g., where an image is projected through acrylic and/or glass, and the image is then backlit with LEDs).

Some or all of the operations performed by the computer 1202 according to the computer program 1210 instructions may be implemented in a special purpose processor 1204B. In this embodiment, some or all of the computer program 1210 instructions may be implemented via firmware instructions stored in a read only memory (ROM), a programmable read only memory (PROM) or flash memory within the special purpose processor 1204B or in memory 1206. The special purpose processor 1204B may also be hardwired through circuit design to perform some or all of the operations to implement the present invention. Further, the special purpose processor 1204B may be a hybrid processor, which includes dedicated circuitry for performing a subset of functions, and other circuits for performing more general functions such as responding to computer program 1210 instructions. In one embodiment, the special purpose processor 1204B is an application specific integrated circuit (ASIC) or a field programmable gate array.

The computer 1202 may also implement a compiler 1212 that allows an application or computer program 1210 written in a programming language such as C, C++, Assembly, SQL, PYTHON, PROLOG, MATLAB, RUBY, RAILS, HASKELL, or other language to be translated into processor 1204 readable code. Alternatively, the compiler 1212 may be an interpreter that executes instructions/source code directly, translates source code into an intermediate representation that is executed, or that executes stored precompiled code. Such source code may be written in a variety of programming languages such as JAVA, JAVASCRIPT, PERL, BASIC, etc. After completion, the application or computer program 1210 accesses and manipulates data accepted from I/O devices and stored in the memory 1206 of the computer 1202 using the relationships and logic that were generated using the compiler 1212.

The computer 1202 also optionally comprises an external communication device such as a modem, satellite link, Ethernet card, or other device for accepting input from, and providing output to, other computers 1202.

In one embodiment, instructions implementing the operating system 1208, the computer program 1210, and the compiler 1212 are tangibly embodied in a non-transitory computer-readable medium, e.g., data storage device 1220, which could include one or more fixed or removable data storage devices, such as a zip drive, floppy disc drive 1224, hard drive, CD-ROM drive, tape drive, etc. Further, the operating system 1208 and the computer program 1210 are comprised of computer program 1210 instructions which, when accessed, read and executed by the computer 1202, cause the computer 1202 to perform the steps necessary to implement and/or use the present invention or to load the program of instructions into a memory 1206, thus creating a special purpose data structure causing the computer 1202 to operate as a specially programmed computer executing the method steps described herein. Computer program 1210 and/or operating instructions may also be tangibly embodied in memory 1206 and/or data communications devices 1230, thereby making a computer program product or article of manufacture according to the invention. As such, the terms “article of manufacture,” “program storage device,” and “computer program product,” as used herein, are intended to encompass a computer program accessible from any computer readable device or media.

Of course, those skilled in the art will recognize that any combination of the above components, or any number of different components, peripherals, and other devices, may be used with the computer 1202.

Generally, these components all comprise logic and/or data that is embodied in/or retrievable from device, medium, signal, or carrier, e.g., a data storage device, a data communications device, a remote computer or device coupled to the computer via a network or via another data communications device, etc. Moreover, this logic and/or data, when read, executed, and/or interpreted, results in the steps necessary to implement and/or use the present invention being performed.

Although the terms “user computer”, “client computer”, and/or “server computer” are referred to herein, it is understood that such computers 1002 and 1006 may be interchangeable and may further include thin client devices with limited or full processing capabilities, portable devices such as cell phones, notebook computers, pocket computers, multi-touch devices, and/or any other devices with suitable processing, communication, and input/output capability.

Of course, those skilled in the art will recognize that any combination of the above components, or any number of different components, peripherals, and other devices, may be used with computers 1200, 1202. Further, as described above, the client 1002 or server computer 1006 may comprise a thin client device or a portable device that has a multi-touch-based display.

Example software used includes, but is not limited to, Adafruit_ADS1x15, Marko Pinteric's MCP4728.

### Advantages and Improvements

Current smart bandage for wound healing are limited to using electric field or limited number of chemicals. The present disclosure reports on a device design that can deliver multiple ions and biomolecules using an electric field. Advantages of this device include closed loop control of sensing and actuation, accelerating wound healing through dry delivery of ions and biomolecules, and programmable wireless actuation of device to deliver over several days.

In one or more examples, the bandages are 3D printed PDMS-based devices capable of delivery of ions and biomolecules through cation-selective ion pumps. Selective ion pumps in the device facilitate the delivery of different ions and biomolecules. Use of 3D printers for fabrication of these devices allows us to manipulate the number of channels in the device. Custom-made programmable PCB boards on each device allows for controlling delivery time and dosage. The use of custom-built cameras for fluorescence imaging may be applied to obtain visual data that allows for closed-loop control.

### Protocols Used for the Data Presented Herein

### Hydrogel-Filled Capillary Preparation

In order to deliver ions such as H+, the four silica capillaries inserted in each device are filled with a polyanion hydrogel that selectively conducts cations. The hydrogel recipe requires 1 M AMPSA, 0.4 M PEGDA, and 0.05 M photoinitiator (12959) concentrations to result in a hydrogel with a low swelling ratio (12%) and good conductivity (8.8=0.1 S/m). A several-centimeter length of the silica tubing (inner diameter 100 μm, outer diameter 375 μm) used for the capillaries is etched with NaOH and further treated with silane A174 to prevent the hydrogel from swelling out of the capillary after UV crosslinking for 5 min at 8 mW/cm². Following UV curing, the capillary tubes are cut into 5 mm pieces. This designated length allows for the easy insertion of capillaries into the device, with minimal cutting required to get the capillaries down to 2 to 3 mm. Cutting the capillary tubing into small lengths beforehand also allows each piece to be checked for electrical connection and loaded with H+. The capillaries are first soaked in a 0.1 M HCl solution for several hours. Both ends of the capillary are then placed in electrolyte wells made from PDMS on a glass slide. 0.5 mm diameter Ag and AgCl wires are used as WE and RE, respectively, at the two ends of each capillary to be loaded. The wells are filled with 0.1 M HCl (source solution) at the WE end and 0.01 M KCl (target solution) at the RE/counter electrode (CE) end. +0.8 V is then applied at the WE, with this level of voltage being selected to avoid water splitting. When the voltage is applied for 5 min, a clean steady-state current is obtained when there are no hydrogel issues. Typical steady-state current measured when loading the capillaries with H+ is 6 μA.

### Animal Experiments

C57B6 males, wildtype mice (32-32 weeks old, 30-35 g) were used. They were acclimated and supplied with DietGel 93M (ClearH2O) and soaked chow to maintain the body weight for one week before the experiment started. The mice were weighted and shaved 1-3 days prior to the surgery. On the day of surgery (Day 0), the animals were anesthetized, saline and analgesics were injected, and the back skin was prepared with betadine and alcohol washes. One wound was created on the side of the spine by suturing silicon splint rings (16 mm outer and 10 mm inner diameters) on the intended location. A 6 mm biopsy punch generated a full-thickness, excisional wound with the silicon splint to control wound contraction on each mouse. The wounds were covered with a vapor permeable secondary dressing (such as Tegaderm) to secure the devices. For the treatment group, devices deliver H+ continuously for a specified duration to reach a recommended target dose. Daily examination of the wounds, dressings and the device functionality are performed for a total of four days starting the surgery day (Day 0). The wound tissue are harvested on Day 3 for IHC staining.

### Histology

At the end of each experiment, after euthanizing the mice, wounds were excised and placed into a paraformaldehyde solution to fix for 24 h. Next, the fixed tissues were processed in a tissue processor for FFPE tissue histology. During processing, the tissues were dehydrated and impregnated with paraffin wax to preserve the tissue structure. Processed tissues were then embedded into paraffin blocks and cut into 5 μm thick sections using a microtome, and the sections were placed onto glass slides. After some drying time, these sections were used for IHC staining to determine the M1/M2 ratio.

### Macrophage IHC Staining

We stained both M1 and M2 macrophages to get an accurate cell count. 4′, 6-diamidino-2-phenylindole (DAPI) is used to mark the nuclei, F4/80 is used to show all macrophages, and iNOS is used to stain M1 while CD206 is used to stain M2. Only those cell nuclei (labeled with DAPI) that overlapped/surrounded by both markers were considered as macrophages, i.e., M1 (iNOS+F4/80+DAPI) and M2 (CD206+F4/80+DAPI). The IHC staining of formalin-fixed, paraffin-embedded tissue sections were achieved over two days as follows:

### Day 1

1. Deparaffinize slides: Place slides in 2 washes of Xylene for 10 min each, 1 wash of Xylene/ETOH for 4 min, 2 washes of 100% EtOH, 2 washes of 95% EtOH, 2 washes of 90% EtOH, 1 wash of 70% EtOH, and 1 wash of 50% ETOH for 4 min per wash. Pre-warm Sodium Citrate pH 6.0 in the rice cooker after the Xylene washes.

2. Wash/tilt slides 3× in 1×PBS (or TBS)+0.1% Tween-20 for 5 min each time.

3. Steam slides in Sodium Citrate Buffer (pH 6.0)+0.05% Tween-20 inside of rice cooker for 30 min. Cool slides to room temp in 1×PBS (or TBS) or H2O for 10-15 min (keep wet).

4. Wash/tilt slides 2×5 min in 1×PBS (or TBS)+0.1% Tween.

5. Use a PAP/Immuno-pen to outline a hydrophobic barrier for incubating tissue.

6. Apply Permeabilization/Blocking Buffer for 2 h in a humidified slide box.

7. Apply primary antibody diluted in Antibody dilution buffer and incubate overnight at 4° C. in a humidified chamber with primary antibodies: Rat anti-F4/80 (dilution 1:50; MCA497G, BIO-RAD, Hercules, CA), Rabbit anti-iNOS (dilution 1:100; PA3-030A, Thermo Fisher Scientific) and Goat anti-CD206 (dilution 1:100; PA5-46994, Thermo Fisher Scientific).

### Day 2

1. Wash the slides 3× in 1×PBS or TBS (+0.1% Tween) for 5 min each.

2. In the dark, apply corresponding Alexa Fluor-conjugated secondary antibodies: Donkey Anti rat-AlexaFluor 488, Donkey Anti rabbit-AlexaFluor 647, and Donkey Anti goat-AlexaFluor 568, dilution 1:200, Thermo Fisher Scientific), for sections from step 8 (on Day 1), and incubate for 1 h and 30 min. During the incubation time, make sure slides are light protected from this step.

3. Wash/tilt 3×5 min in 1×PBS/TBS (+0.1% Tween).

4. Dilute sufficient DAPI stock by 1:1000 in DI H2O and place on slides for min.

5. Wash/tilt 3×5 min in 1×PBS or TBS.

6. Drain excess PBS using paper towels, while carefully avoiding tissue.

7. Apply ˜22.5 L of anti-fade mounting media (SlowFade Mountant; S36936, Thermo Fisher Scientific) and gently add cover slips over sections to carefully not make bubbles. Use tweezers to guide the coverslip slowly.

8. Put nail polish around the edges and let dry for 15 min overnight.

### REFERENCES

The following references are incorporated by reference herein.
1. Singer, A. J. & Clark, R. A. Cutaneous wound healing. New Engl. J. Medicine 341, 738-746, DOI: 10.1056/NEJM199909023411006 (1999). PMID: 10471461, https://doi.org/10.1056/NEJM199909023411006.
2. Baum, C. L. & Arpey, C. J. Normal cutaneous wound healing: Clinical correlation with cellular and molecular events. Dermatol. Surg. 31, 674-686, DOI: https://doi.org/10.1111/j.1524-4725.2005.31612 (2005). https://onlinelibrary.wiley. com/doi/pdf/10.1111/j.1524-4725.2005.31612.
3. Liu, Z.-J. & Velazquez, O. C. Hyperoxia, endothelial progenitor cell mobilization, and diabetic wound healing. Antioxidants & Redox Signal. 10, 1869-1882, DOI: 10.1089/ars.2008.2121 (2008). PMID: 18627349, https://doi.org/10.1089/ars.2008. 2121.
4. Kumar, A. & Brockes, J. P. Nerve dependence in tissue, organ, and appendage regeneration. Trends neurosciences 35, 691-699 (2012).
5. Nieto-Diaz, M. et al. Deer antler innervation and regeneration. Front. Biosci. 17, 1389-1401 (2012).
6. Payzin-Dogru, D. & Whited, J. L. An integrative framework for salamander and mouse limb regeneration. Int. J. Dev. Biol.
62, 393-402 (2018).
7. Vivien, C. J., Hudson, J. E. & Porrello, E. R. Evolution, comparative biology and ontogeny of vertebrate heart regeneration. NPJ Regen. medicine 1, 1-14 (2016).
8. Pinto, A. R., Godwin, J. W. & Rosenthal, N. A. Macrophages in cardiac homeostasis, injury responses and progenitor cell mobilisation. Stem Cell Res. 13, 705-714, DOI: https://doi.org/10.1016/j.scr.2014.06.004 (2014). Heart Regeneration and Rejuvenation.
9. Forbes, S. J. & Rosenthal, N. Preparing the ground for tissue regeneration: from mechanism to therapy. Nat. medicine 20, 857-869 (2014).
10. Anders, H.-J. Immune system modulation of kidney regeneration-mechanisms and implications. Nat. Rev. Nephrol. 10, 347-358 (2014).
11. Gensel, J. C., Kigerl, K. A., Mandrekar-Colucci, S. S., Gaudet, A. D. & Popovich, P. G. Achieving cns axon regeneration by manipulating convergent neuro-immune signaling. Cell tissue research 349, 201-213 (2012).
12. Mescher, A. L. & Neff, A. W. Regenerative capacity and the developing immune system. Regen. medicine I 39-66 (2005).
13. Chen, L., Cheng, L., Chen, T., Zhang, Y. & Zhang, J. Macrophage polarization in skin wound healing: Progress in biology and therapeutics. J. Shanghai Jiaotong Univ. (Science) 27, 264-280 (2022).
14. Zhao, M. Electrical fields in wound healing—an overriding signal that directs cell migration. Semin. Cell Dev. Biol. 20, 674-682, DOI: https://doi.org/10.1016/j.semcdb.2008.12.009 (2009). Regenerative Biology and Medicine: II and Patterning and Evolving the Vertebrate Forebrain.
15. Reid, B. & Zhao, M. The electrical response to injury: Molecular mechanisms and wound healing. Adv. Wound Care 3, 184-201, DOI: 10.1089/wound.2013.0442 (2014). https://doi.org/10.1089/wound.2013.0442.
16. Zhao, M. et al. Electrical signals control wound healing through phosphatidylinositol-3-oh kinase-γ and pten. Nature 442, 457-460 (2006).
17. Godwin, J. W., Pinto, A. R. & Rosenthal, N. A. Macrophages are required for adult salamander limb regeneration. Proc. Natl. Acad. Sci. 110, 9415-9420, DOI: 10.1073/pnas. 1300290110 (2013). https://www.pnas.org/doi/pdf/10.1073/pnas.
1300290110.
18. Lucas, T. et al. Differential roles of macrophages in diverse phases of skin repair. The J. Immunol. 184, 3964-3977, DOI: 10.4049/jimmunol.0903356 (2010). https://www.jimmunol.org/content/184/7/3964.full.pdf.
19. Kar, A. et al. Wearable and implantable devices for drug delivery: Applications and challenges. Biomaterials 283, 121435, DOI: https://doi.org/10.1016/j.biomaterials.2022.121435 (2022).
20. Proctor, C. M. et al. An electrocorticography device with an integrated microfluidic ion pump for simultaneous neural recording and electrophoretic drug delivery in vivo. Adv. Biosyst. 3, 1800270, DOI: https://doi.org/10.1002/adbi.201800270 (2019). https://onlinelibrary.wiley.com/doi/pdf/10.1002/adbi.201800270.
21. Jonsson, A. et al. Therapy using implanted organic bioelectronics. Sci. Adv. 1, e1500039, DOI: 10.1126/sciadv.1500039 (2015). https://www.science.org/doi/pdf/10.1126/sciadv.1500039.
22. Arbring Sjöström, T. et al. A decade of iontronic delivery devices. Adv. Mater. Technol. 3, 1700360, DOI: https:
//doi.org/10.1002/admt.201700360 (2018). https://onlinelibrary.wiley.com/doi/pdf/10.1002/admt.201700360.
23. Jakešová, M. et al. Wireless organic electronic ion pumps driven by photovoltaics. npj Flex. Electron. 3, 1-6 (2019).
24. Liang, Y., Offenhäusser, A., Ingebrandt, S. & Mayer, D. Pedot: pss-based bioelectronic devices for recording and modulation of electrophysiological and biochemical cell signals. Adv. Healthc. Mater. 10, 2100061, DOI: https://doi.org/10.1002/adhm.202100061 (2021). https://onlinelibrary.wiley.com/doi/pdf/10.1002/adhm.202100061.
25. Xu, G. et al. Battery-free and wireless smart wound dressing for wound infection monitoring and electrically controlled on-demand drug delivery. Adv. Funct. Mater. 31, 2100852, DOI: https://doi.org/10.1002/adfm.202100852 (2021). https:
//onlinelibrary.wiley.com/doi/pdf/10.1002/adfm.202100852.
26. Laughner, J. I. et al. A fully implantable pacemaker for the mouse: From battery to wireless power. PLOS ONE 8, 1-8, DOI: 10.1371/journal.pone.0076291 (2013).
27. Long, Y. et al. Effective wound healing enabled by discrete alternative electric fields from wearable nanogenerators. ACS Nano 12, 12533-12540, DOI: 10.1021/acsnano.8b07038 (2018). PMID: 30488695, https://doi.org/10.1021/acsnano. 8b07038.
28. Luo, R., Dai, J., Zhang, J. & Li, Z. Accelerated skin wound healing by electrical stimulation. Adv. Healthc. Mater. 10, 2100557, DOI: https://doi.org/10.1002/adhm.202100557 (2021). https://onlinelibrary.wiley.com/doi/pdf/10.1002/adhm.
202100557.
29. Wang, K. et al. A platform to study the effects of electrical stimulation on immune cell activation during wound healing. Adv. Biosyst. 3, 1900106, DOI: https://doi.org/10.1002/adbi.201900106 (2019). https://onlinelibrary.wiley.com/doi/pdf/10. 1002/adbi.201900106.
30. Montgomery, K. L. et al. Wirelessly powered, fully internal optogenetics for brain, spinal and peripheral circuits in mice. Nat, methods 12, 969-974 (2015).
31. Wentz, C. T. et al. A wirelessly powered and controlled device for optical neural control of freely-behaving animals. J. Neural Eng. 8, 046021, DOI: 10.1088/1741-2560/8/4/046021 (2011).
32. Pansodtee, P. et al. The multi-channel potentiostat: Development and evaluation of a scalable mini-potentiostat array for investigating electrochemical reaction mechanisms. PLOS ONE 16, 1-14, DOI: 10.1371/journal.pone.0257167 (2021).
33. Forsyth, L., Witton, J., Brown, J., Randall, A. & Jones, M. In vitro and in vivo recording of local field potential oscillations in mouse hippocampus. Curr. Protoc. Mouse Biol. 2, 273-294, DOI: https://doi.org/10.1002/9780470942390.mo120089 (2012). https://currentprotocols.onlinelibrary.wiley.com/doi/pdf/10.1002/9780470942390.mo 120089.
34. Fan, D. et al. A wireless multi-channel recording system for freely behaving mice and rats. PloS one 6, e22033 (2011).
35. Mendrela, A. E. et al. A miniature headstage for high resolution closed-loop optogenetics. In 2017 IEEE Biomedical Circuits and Systems Conference (BioCAS), 1-4, DOI: 10.1109/BIOCAS.2017.8325187 (2017).
36. Nguyen, C. M. et al. Topical Fluoxetine as a Novel Therapeutic That Improves Wound Healing in Diabetic Mice. Diabetes 68, 1499-1507, DOI: 10.2337/db18-1146 (2019). https://diabetesjournals.org/diabetes/article-pdf/68/7/1499/526749/db181146.pdf.
37. Yoon, D. J. et al. Topical fluoxetine as a potential nonantibiotic adjunctive therapy for infected wounds. The J. investigative dermatology 141, 1608-1612 (2021).
38. Alhakamy, N. A. et al. Fluoxetine ecofriendly nanoemulsion enhances wound healing in diabetic rats: In vivo efficacy assessment. Pharmaceutics 14, DOI: 10.3390/pharmaceutics14061133 (2022).
39. Controlling pH by electronic ion pumps to fight fibrosis

Anne Géraldine Guex a, b, *, David J. Poxson c, Daniel T. Simon c, Magnus Berggren c, Giuseppino Fortunato b, 1, RenéM. Rossi b, Katharina Maniura-Weber a, Markus Rottmar
40. Deleted
41. Singhal, M. & Kalia, Y. N. Iontophoresis and Electroporation, 165-182 (Springer Japan, Tokyo, 2017).
42.Bakshi, P., Vora, D., Hemmady, K. & Banga, A. Iontophoretic skin delivery systems: Success and failures. Int. J. Pharm. 586, 119584, DOI: https://doi.org/10.1016/j.ijpharm.2020.119584 (2020).
43. Kalia, Y. N., Naik, A., Garrison, J. & Guy, R. H. Iontophoretic drug delivery. Adv. Drug Deliv. Rev. 56, 619-658, DOI: https://doi.org/10.1016/j.addr.2003.10.026 (2004). Breaking the Skin Barrier.
44.Dechiraju, H. et al. On-chip on-demand delivery of k+ for in vitro bioelectronics. AIP Adv. 12, 125205, DOI: 10.1063/5. 0129134 (2022). https://doi.org/10.1063/5.0129134.
45. Ellison, G., Straumfjord, J., Jon V & Hummel, J. P. Buffer Capacities of Human Blood and Plasma. Clin. Chem. 4, 452-461, DOI: 10.1093/clinchem/4.6.452 (1958). https://academic.oup.com/clinchem/article-pdf/4/6/452/32872607/clinchem0452. pdf.
46. Guex, A. G. et al. Controlling ph by electronic ion pumps to fight fibrosis. Appl. Mater. Today 22, 100936, DOI: https://doi.org/10.1016/j.apmt.2021.100936 (2021).
47 Microchip Technology Inc., USA.ATtiny85V.ATtiny25 45 85 Automotive Specifi-cation at 1.8V Datasheet (2013) https://ww1.microchip.com/downloads/en/DeviceDoc/Atmel-7669-ATtiny25-45-85-Appendix-B-Automotive-Specification-at-1.8V_Datasheet.pdf.
48. Schneider, L. A., Korber, A., Grabbe, S. & Dissemond, J. Influence of ph on wound-healing: a new perspective for wound-therapy? Arch. dermatological research 298, 413-420 (2007).

### CONCLUSION

This concludes the description of the preferred embodiment of the present invention. The foregoing description of one or more embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

1. A device, comprising:
a wearable ion pumping system pumping ions to and from a treatment site so as to deliver and measure a dose of the ions treating the treatment site.

2. The device of claim 1, wherein the wearable ion pumping system further comprises a control circuit activating the pumping and measuring the dose.

3. The device of claim 2, wherein the wearable ion pumping system further comprises:
a housing comprising one or more reservoirs storing a fluid comprising the ions;
a system of conduits connected to the housing, the conduits loaded with a fluid between the reservoir and the treatment site; and
a plurality of electrodes electrically connected to the fluid and the control circuit, wherein in the control circuit activates the pumping by applying one or more voltages to the fluid via the electrodes.

4. The device of claim 3, wherein the control circuit comprising a potentiostat applying the one or more voltages and sensing one or more currents flowing through the treatment site.

5. The device of claim 3, wherein:
the electrodes each comprise a pin comprising a first end electrically coupled to a wire,
the housing consists essentially of a polymer and comprises:
molded cavities comprising the reservoirs;
through holes through which the pins are inserted, so that the first end of each of the pins electrically connects to the wire in a different one of the reservoirs; and
mounts comprising openings through which each of the conduits are inserted and mounting a first conduit end of each of the conduits in fluidic connection with a different one of the reservoirs, so that a second end of the each of the conduits can be in physical contact with the treatment site; and
the device further comprises a printed circuit board soldered to a second end of the pins, wherein the printed circuit board is electrically connected to the control circuit.

6. The device of claim 5, wherein:
the housing comprises:
a top (printed circuit board side) part comprising the molded cavities and the through holes and physically attached to the printed circuit board side via the pins; and
a bottom (treatment site side) part comprising the mounts and a lid enclosing the molded cavities to form the reservoirs; and
the conduits comprise capillaries.

7. The device of claim 5, wherein the polymer consists essentially of PDMS.

8. The device of claim 5, wherein:
the reservoirs comprise one or more first reservoirs and one or more second reservoirs;
the ions comprise first polarity ions and second polarity ions; and
the control circuit applies:
the voltage comprising a first polarity bias to the fluid via the electrodes in the first reservoirs to drive the first polarity ions to the treatment site through a first one of the conduits; and
the voltage comprising a second polarity bias to the fluid via the electrodes in the second reservoirs to pull the second polarity ions from the treatment site through a second one of the conduits, to maintain charge balance.

9. The device of claim 8, wherein the control circuit measures a current comprising the ions to determine the dose of ions delivered to the treatment site.

10. The system of claim 5, wherein the wires are embedded in the polymer and in fluidic connection with the fluid in the reservoir.

11. The device of claim 10, wherein the wires are connected to the pins via a conductive paste and the wires extend across a majority of a length or width of the reservoir.

12. The device of claim 3, wherein the control circuit comprises multiple channels, each of the channels connected to a different one of the voltages and comprising one of the conduits delivering the ions in response to the one of the voltages and a current sense resistor in series with the voltage for sensing the current in the each of the channels.

13. The device of claim 3, further comprising an electrical connector, wherein the control circuit is remotely connected to the electrodes with wiring via a break out board and the electrical connector.

14. The device of claim 3, wherein the ions comprise H+ ions or protons.

15. The device of claim 14, wherein the fluid in the conduits comprises a hydrogel and the fluid in the reservoirs comprises a solution comprising the ions.

16. The device of claim 2, wherein the ion pumping system comprises a microfluidic system pumping a fluid comprising the ions.

17. The device of claim 2, wherein the ions control a pH of the treatment site.

18. The device of claim 17, wherein the control circuit controls the ions to lower the pH so as to transition macrophages in the treatment site to an anti-inflammatory pro-reparative phonotype (away from an inflammatory phenotype) early in the treatment cycle.

19. A system comprising the device of claim 2 coupled to a camera forming images of the wound, wherein the control circuit provides closed loop control of the pumping based on healing of the wound observed in the images.

20. (canceled)

21. (canceled)

22. (canceled)

23. The device of claim 1, further comprising a bandage, dressing, adhesive, or patch attaching the device to the treatment site and covering the treatment site, wherein the treatment site comprises a wound and the ion pumping system pumps biomolecules comprising the ions accelerating healing of the wound.

24. (canceled)
